(19) United States
(12) Patent Application Publication
(10) Pub. No.: US 2023/0277613 A1
(43) Pub. Date: Sep. 7, 2023

(54) Polysaccharide-Rich Bombax Costatum Flower Extract

(71) Applicant: Laboratoires Expanscience, Paris La Defense Cedex (FR)

(72) Inventors: Sophie LECLERE-BIENFAIT, Dreux (FR); Stephanie BREDIF, Croisilles (FR)

(73) Assignee: Laboratoires Expanscience, Paris La Defense Cedex (FR)

(21) Appl. No.: 18/017,011

(22) PCT Filed: Jul. 26, 2021
(86) PCT No.: PCT/FR2021/051394

§ 371 Date: Jan. 19, 2023

(30) Foreign Application Priority Data
Jul. 24, 2020 (FR) 2007834

(51) Int. Cl. A61K 36/185; A61K 8/9789; A61Q 19/08; A61Q 17/00; A61P 31/02; A61P 17/00; A61P 17/10; A61Q 15/00

(52) U.S. Cl. CPC A61K 36/185; A61K 8/9789; A61Q 19/08; A61Q 17/00; A61P 31/02; A61P 17/00; A61P 17/10; A61Q 15/00; A61K 2236/15; A61K 2236/31; A61K 2236/331; A61K 2236/53; A61K 2236/51

(57) ABSTRACT

The invention relates to an extract from the flowers, preferably from the calyxes, of Bombax costatum, to a process for preparing same and to the extract obtained by said process. The invention also relates to a composition comprising such an extract, the composition advantageously being a cosmetic, pharmaceutical or dermatological composition. The invention also relates to such a composition or such an extract for the use thereof in preventing or treating disorders or pathological conditions of the skin, the mucous membranes or the hair and nails, as well as the imbalance or disorders associated with the imbalance of the microbiota of the skin, the mucous membranes, the hair, the nails and associated skin appendages. Finally, the invention relates to a cosmetic care method for the skin, the hair and nails or the mucous membranes, with a view to improving the condition or the appearance thereof, which method consists in administering such a composition or such an extract.

The invention relates to a Bombax costatum flower extract, said extract being rich in polysaccharides. The invention also relates to a cosmetic, pharmaceutical or dermatological composition comprising such an extract. The invention also relates to a process for extracting a polysaccharide-rich Bombax costatum flower extract, as well as the extract that can be obtained by said process. The invention also relates to such a composition or such an extract for the use thereof in preventing or treating disorders or pathological conditions of the skin, the mucous membranes or skin appendages, and in preventing or treating vascular disorders. The invention finally relates to a cosmetic care method for the skin, skin appendages or mucous membranes, in view of improving the condition or the appearance thereof, which method consists in administering such a composition or such an extract.

Bombax Costatum

The Bombax costatum, belongs to the bombacaceae family (APG: Malvaceae). The Bombax costatum is also called Bombax andrieui or Bombax houardii. The Bombax costatum is more commonly called Kapok tree with red flowers, cheese tree, red kapok tree, false kapok tree, forest kapok tree, Voaka (in the Moré language), or Boumbou (in the Jula language).

Bombax is a pantropical genus comprising 8 species: 2 in Africa, 5 in Asia, and 1 in Oceania, present as far as the Solomon Islands. In the past, the delimitation of the Bombax genus was much wider. Bombax costatum is sometimes considered conspecific with Bombax buonopozense.

Uses of Bombax costatum

In general, in Africa, the Bombax costatum is a fairly common food tree: the leaves are dried and eaten like those of the Baobab; the flowers, especially the calyxes, are also eaten.

In Burkina Faso the bark is used to stain teeth red. The flowers are commonly harvested for the fleshy calyx which is cooked and eaten as a vegetable. The leaves are also eaten as a vegetable. The immature fruits and sometimes the flowers are added as thickeners in sauces. Young immature fruits are also used in the preparation of a drink. The seed oil is edible. The flowers are highly valued as honey flowers.

Several parts of the tree are used in traditional medicine against different diseases. Maceration of the powdered root is eaten in sauce or applied as a bath against epilepsy. Bark preparations are applied to wounds to promote healing.

In Senegal and Sierra Leone, diuretic properties are attributed to the stem and root bark. The bark is also used to prepare a medicine against trichomoniasis, amoebiasis and other forms of dysentery. A bath in a stem bark extract is taken against insanity. Powdered stem bark is used in the composition of a medicine applied as a fumigant against headache. To treat headache or toothache, a bark compress can be placed on the head.

The leaves are prescribed with other medicinal plants to treat leucorrhoea and diarrhoea. An extract of crushed leaves is taken as a drink against problems during childbirth. A bath in an extract of crushed leaves is taken repeatedly against convulsions. A tea of dried leaves is taken or applied to the body against measles. A decoction of the leaves and stem or root bark is taken as a drink in cases of severe swelling. A decoction of leaves and young twigs is drunk to treat jaundice. A leaf decoction is also given to children to drink against rickets.

Different parts of the plant are used to promote lactation and as a tonic against fatigue. The skin is rubbed with leaves mixed with shea butter against leprosy.

In Mali a decoction of the bark and leaves and parts of other plants is taken against menstruation disorders. The leaves are emollient and a warm bath in a leaf decoction may be prescribed for patients with fever, especially children. The leaves are also used in hookworm treatments and the flowers in tapeworm treatments.

All these data were taken from the ethnobotanical report written by Dr. Lassina SANOU, Colonel of Waters and Forests. He works for the Ministry of Environment and Sustainable Development at the National Tree Seed Centre (CNRS) of Burkina Faso, based in Ouagadougou (December 2014).

Polysaccharides

Polyosides or polysaccharides or glycans are arbitrarily defined as high molecular weight polymers resulting from the condensation of a large number of oses or sugars. They participate in different aspects in the life and even survival of plants. For example, they are responsible for the rigidity of the cell walls of higher plants (celluloses, hemi-cellulose, lignins, . . . ), they are forms of energy storage (starch) and can protect tissues against dehydration due to their hydrophilic power . . .

We distinguish:
Homogeneous polysaccharides (condensation of the same ose); and
Heterogeneous polysaccharides (hexoses, pentoses, ose ether, . . . ), and the 2 categories can be linear or ramified.

THE MICROBIOTA and THE MICROBIOME

The skin microbiota is all the microorganisms (bacteria, viruses, fungi, . . . ) residing on and in the skin. The microbiota is present even in the dermis and adipose tissue and eccrine glands (Nakatsuji and al 2013). The microbiota should not be confused with the microbiome which is the set of genes (genome of bacteria).

There are 2 main types of flora:
the resident flora composed of commensal germs living at the expense of their host without causing damage; and
the transient flora composed of saprophytic germs (harmless) and opportunistic pathogens.

In the skin, there are 1012 bacterial cells (106/cm²) for 3.1010 human cells (ratio 33:1 vs gastrointestinal system: ratio 25:1); 500 different germs; 1 resident or transient flora; and 4 predominant Phyla.

There are many skin/microbiota interactions:
Phenomena of symbiosis: the skin represents an environmental niche, a reservoir of nutrients for certain bacteria.

Bacteria can stimulate immune defenses; and the skin in response can develop a veritable anti-microbial shield that inhibits the growth of pathogens and stimulates the production of anti-microbial molecules, which contributes to maintaining a good state of skin health. But depending on the context, the same bacterium can be symbiotic or pathobiontic. Symbionts lead to physiological inflammation, barrier protection, surveillance/tolerance (PAMs) by the skin and its innate immunity, absence of virulence, and sequestration of TLRs (Toll-like receptors). Symbionts live in biofilms. It sometimes occurs for various intrinsic and/or environmental reasons, a loss of control of these symbionts which then become pathobionts. The virulence factors of the microbiota increase, as well as inflammatory phenomena and the activation of the immune system. Commensal bacteria are sometimes eradicated.

Under so-called normal skin conditions, host immunity maintains the balance of the microbiota. A deficiency in the immune system leads to dysbiosis and specific skin pathological conditions: atopic dermatitis, acne, psoriasis. An increase is seen in colonization of the skin by P. acnes during acne, by Staphyloccocus aureus during atopic dermatitis, by the Firmicutes family during psoriasis (and decrease in actinobacteria), by Malassezia furfur during seborrheic dermatitis.

### DESCRIPTION OF THE INVENTION

The Applicant has discovered that the extracts of Bombax costatum flower, preferably of Bombax costatum calyxes, have cosmetic and dermatological properties never before described.

The invention relates to a polysaccharide-rich extract from the Bombax costatum flowers, preferably from the Bombax costatum calyxes.

In botany, the flower is the part of the plant comprising, from the outside to the inside, when complete:
the calyx, formed by all the sepals;
the corolla, formed by all the petals;
the androecium, that is to say all the stamens (male part), which produces the pollen; and
the gynoecium or pistil, formed by all the carpels (female part).

In the context of the present invention, the term “flower” comprises at least the corolla and the calyx. The flower can also be complete, that is to say comprise the calyx, the corolla, the androecium, and the gynoecium or pistil.

The extract according to the invention is preferably a Bombax costatum calyx extract.

“Polysaccharide-rich extract” means an extract comprising mainly or essentially polysaccharides, that is to say that the majority compounds are polysaccharides.

The extract according to the invention thus advantageously comprises at least 15% by weight of polysaccharides, more advantageously at least 20% by weight, preferably at least 30% by weight, more advantageously at least 50% by weight, relative to the total dry extract weight. The extract according to the invention thus advantageously comprises from 15% to 65% by weight of polysaccharides, more advantageously from 20% to 65% by weight, preferably from 30% to 65% by weight, more advantageously from 50% to 65% by weight, relative to the total weight of the dry extract. The percentages are expressed relative to the total weight of said dry extract (before any addition of a drying medium) and the assay is carried out according to the sulfuric phenol method (Dubois method) or according to the anthrone method—spectro-colorimetric assay of total sugars.

According to the present invention, the polysaccharides present in the extract have an apparent molecular mass comprised between 1500 kDa and 6000 kDa, advantageously between 3000 kDa and 6000 kDa (determination by Gas Phase Chromatography).

Advantageously, the polysaccharides of the extract according to the invention comprise monosaccharides and derivatives selected from the group consisting of galactose, rhamnose, galacturonic acid, glucuronic acid, and mixtures thereof. In the context of the present invention, galactose means D-galactose and L-galactose. Similarly, the term rhamnose means D-rhamnose and L-rhamnose.

Advantageously, the polysaccharides of the extract according to the invention comprise a mixture of galactose, rhamnose, galacturonic acid, and glucuronic acid.

Advantageously, the polysaccharides of the extract according to the invention comprise (% by weight relative to the total weight of all the sugars (monosaccharides) present):
From 10% to 16% Galactose;
From 10% to 17% Rhamnose;
From 10% to 17% galacturonic acid; and
From 3% to 7% glucuronic acid.

The extract according to the invention is advantageously substantially free of polyphenols. Typically, the extract according to the present invention comprises less than 1% by weight of polyphenols, advantageously less than 0.5% by weight of polyphenols, relative to the total weight of the dry extract.

In the context of the present invention, the polysaccharide-rich extract described above is advantageously obtained by solid/liquid extraction of the flowers, preferably the calyxes, of Bombax costatum, in water. The polysaccharides can then be purified and/or re-solubilized in a suitable solvent in order to guarantee their physical and microbiological stability or dried by processes known to the person skilled in the art.

The invention also relates to a process for preparing a polysaccharide-rich extract from the flowers, preferably from the calyxes, of Bombax costatum, comprising at least one step of solid/liquid extraction in water and under optimal pH, time and temperature conditions, known to the person skilled in the art.

Advantageously according to the invention, the process for preparing a polysaccharide-rich extract from the flowers, preferably from the calyxes, of Bombax costatum, comprises the following successive steps:
a) crushing the flowers, in particular the calyxes, of Bombax costatum;
b) extracting the flowers, in particular the calyxes, crushed in water under optimal time, pH and temperature conditions;
c) separating the solid phase and the liquid phase by decantation, and/or centrifugation and/or precipitation and/or successive filtrations; and
d) optionally, step of bleaching the liquid phase using a suitable adjuvant;
e) optionally, drying the extract obtained in step c) or d); and
f) optionally, stabilizing the polysaccharides in a liquid medium in a solvent suitable for preserving the physico-chemical properties of the extract and controlling microbial growth.

Advantageously according to the invention, the process for preparing a polysaccharide-rich extract from the flowers, preferably from the calyxes, of Bombax costatum, comprises the following successive steps:
a) crushing the flowers, in particular the calyxes, of Bombax costatum;
b) extracting the flowers, in particular the calyxes, crushed in water;
c) separating the solid phase and the liquid phase by decantation, and/or centrifugation and/or precipitation and/or successive filtrations;
d) optionally, step of bleaching the liquid phase using a suitable adjuvant;
e) optionally, drying the extract obtained in step c) or d); and
f) optionally, physically and microbiologically stabilizing the extract obtained in step c), d) or e).

Step a) of crushing the plant can be carried out by methods known to the person skilled in the art, in particular using a knife mill or a hammer mill.

The extraction step b) is preferably carried out in the presence of water. The percentage of crushed plant introduced into the water is advantageously comprised between 2% and 10% w/w and preferably between 2% and 5% w/w, more advantageously 2% w/w. This solid/liquid extraction is preferably carried out at a temperature comprised between 20° C. and 100° C., in particular between 50° C. and 90° C., more particularly between 70° C. and 90° C., typically 90° C.

The extraction time is advantageously comprised between 30 minutes and 4 hours, in particular between 1 hour and 3 hours, advantageously it is approximately 1 hour.

Step c) of separating the solid phase and the liquid phase is carried out by methods known to the person skilled in the art, in particular by decantation, centrifugation and/or successive filtrations and/or purification by precipitation of the polysaccharides using a suitable solvent and preferably ethanol or a saline solution. In particular, during step c), the liquid phase obtained is advantageously purified and concentrated, for example by ultrafiltration and/or sterilizing filtration. Step c) is carried out until a liquid phase is obtained which has perfect clarity and microbiological cleanliness of a degree less than or equal to 100 CFU/g in total germ.

When step d) of bleaching the liquid phase is carried out, this is done by methods known to the person skilled in the art, in particular by adding an adjuvant such as activated carbon or bleaching earths which are suitable and known to the skilled person.

Advantageously according to the invention, the extraction with water leads to an extract that is very poor in polyphenols or substantially devoid of polyphenols (polyphenols typically in trace quantities). The extract according to the invention, preferably obtained by solid/liquid extraction in water, typically has a content of less than 1% by weight, preferably less than 0.5% by weight, relative to the total weight of the dry extract.

Advantageously according to the present invention, the process according to the invention comprises at least one step of specific filtration and/or purification (step c)) and/or bleaching on activated carbon (step d)) which allow(s) to eliminate the remaining polyphenols if necessary. In particular, the process comprises at least one purification step such as ultrafiltration which allows to separate the “small” molecules such as polyphenols from the “large” molecules (size greater than 1 million Daltons) such as polysaccharides. In addition, advantageously, the treatment with activated carbon during the bleaching step d) also contributes to lowering the level of polyphenols of the extract to be advantageously present only in trace quantities at the end of the process according to the present invention.

Advantageously, the polysaccharide-rich extract according to the invention can be stabilized by a drying step e), by processes known to the person skilled in the art.

The drying step e) can, for example, be carried out in the presence of a carrier of the maltodextrin or acacia fiber type (Fibregum® company CNI). The carrier content typically varies according to a ratio ranging from 0% to 80% carrier relative to the percentage of dry matter obtained in the liquid form of the extract.

The extract is preferably dried by freeze-drying or atomization in order to obtain a final powder.

Step f) of physically and microbiologically stabilizing the product obtained in step c), d) or e) is advantageously carried out by partial elimination of the water and replacement with a solvent advantageously selected from vegetable glycerin, glycols, and mixtures thereof, in particular from vegetable glycerin, glycols of vegetable origin and mixtures thereof, more particularly from a propanediol or propylene glycol, in particular 1,3-propanediol, and vegetable glycerin. Advantageously, step f) is carried out in the liquid phase by reducing the quantity of water by evaporation to less than 50% by weight and preferably less than 20% by weight, relative to the total mass of the extract and substitution with a bacteriostatic or bactericidal solvent selected from vegetable glycerin, glycols, and mixtures thereof, in particular from vegetable glycerin, glycols of vegetable origin and mixtures thereof, more particularly from a propanediol or propylene glycol, in particular 1,3-propanediol, and vegetable glycerin. The water/solvent ratio will then be advantageously comprised between the ratios 50/50 and 0/100 (w/w) and preferentially between 30/70 (w/w) and 10/90 (w/p) and advantageously 20/80 (p/p).

Preferably, by way of example, the polysaccharide-rich extract according to the invention can be obtained according to the following process:
a′) Dissolution of the crushed flower at 2% (w/w) in water
b′) Extraction with stirring for 1 hour at 90° C.;
c′) Purification by successive filtration steps;
d′) Sterile filtration; and
e′) Stabilization of the extract obtained by evaporation of water and addition of glycerin to obtain a water/glycerin ratio comprised between 50/50 and 0/100, advantageously between 30/70 and 10/90.

In the rest of the description, “extract according to the invention” will be considered to designate the extract as such, as defined above, or the extract capable of being obtained by the process according to invention as described above. The extract obtainable by the process according to the invention as described above has the same composition as the extract according to the invention as such, as defined above.

The invention also relates to a composition comprising a polysaccharide-rich extract from the flowers, preferably from the calyxes, of Bombax costatum according to the invention and a water/solvent mixture in a water/solvent ratio (v/v) comprised between 50/50 and 0/100, advantageously between 30/70 and 10/90, more advantageously 20/80, said solvent being selected from glycols, vegetable glycerin and mixtures thereof, preferably from glycols of vegetable origin and glycerin vegetable, and preferably from 1,3-propanediol and vegetable glycerin.

Advantageously, the composition comprises from 0.001 to 30% by weight, advantageously 0.001% to 10% by weight, of an extract according to the invention (expressed by weight of dry extract relative to the total weight of the composition) and between 50% and 99.999% by weight, advantageously between 70% and 99.999% by weight, of a water/solvent mixture, relative to the total weight of the composition, the water/solvent ratio being comprised between 50/50 and 0/100, advantageously between 30/70 and 10/90, more advantageously 20/80, and the solvent being selected from glycols, vegetable glycerin and mixtures thereof, preferably from glycols of vegetable origin and vegetable glycerin, and preferentially from 1,3-propanediol and vegetable glycerin. According to this aspect of the invention, the solvent is in a quantity effective for a physical and microbiological stabilizing action of the composition according to the invention and in particular of the extract according to the invention.

The invention also relates to a composition comprising a polysaccharide-rich extract from the flowers, preferably from the calyxes, of Bombax costatum according to the invention, as active ingredient, and where appropriate a suitable excipient. The extract according to the invention is as defined in the paragraphs above concerning the extract as such and those concerning the extract capable of being obtained by the process according to the invention.

The composition is advantageously a cosmetic, pharmaceutical or dermatological composition. Said composition is preferably formulated to be administered by external topical route.

Advantageously, the composition according to the invention comprises from 0.001 to 10%, typically from 0.01 to 5%, by weight of extract according to the invention, the weight of the extract being expressed in dry extract, relative to the total weight of the composition.

The composition according to the invention may also comprise one or more other active ingredients.

The composition according to the invention can be formulated in the form of various preparations suitable for topical administration and include in particular creams, emulsions, milks, ointments, lotions, oils, aqueous or hydro-alcoholic or glycolic solutions, powders, patches, sprays, shampoos, varnishes or any other product for external application.

Depending on its nature (cosmetic, pharmaceutical or dermatological), the composition according to the invention may also comprise at least one cosmetically, pharmaceutically or dermatologically acceptable excipient. In particular, the composition according to the present invention may also comprise at least one cosmetically, pharmaceutically or dermatologically adjuvant known to the person skilled in the art, in particular selected from surfactants, thickeners, preservatives, perfumes, dyes, chemical or mineral filters, moisturizing agents, and thermal waters. A person skilled in the art knows how to adapt the formulation of the composition according to the invention by using his general knowledge.

The optimal dosages and galenic forms of the compositions according to the invention can be determined according to the criteria generally taken into account in the establishment of a pharmacological, dermatological or cosmetic treatment adapted to a patient or to an animal, such as for example the age or body weight of the patient or animal, the severity of his general condition, tolerance to treatment, side effects observed, type of skin.

The invention also relates to an extract according to the invention or a composition according to the invention for the use thereof in preventing and/or treating disorders or pathological conditions of immature, normal or mature/aged skin and/or mucous membranes (gums, periodontium, genital mucous membranes) and/or skin appendages (hair and nails), in particular inflammatory reactions, oxidation reactions, disorders related to radical attacks related or not to pollution, disorders or pathological conditions related to microbial attacks, barrier or homeostasis disorders, aging, in particular chronological and/or actinic aging, disorders or pathological conditions related to mechanical and/or thermal aggressions on the skin and/or mucous membranes and/or skin appendages; more advantageously inflammatory or irritative reactions or pathological conditions or disorders of the barrier or homeostasis of immature, normal or mature/aged skin, skin appendages and/or mucous membranes (gums, periodontium, genital mucous membranes).

The invention also relates to an extract according to the invention or a composition according to the invention for the use thereof in preventing and/or treating imbalances and/or disorders related to the imbalance of the microbiota of immature, normal or mature/aged skin and/or mucous membranes (gums, periodontium, genital mucous membranes) and/or skin appendages and/or annexes thereof.

Indeed, the extract according to the invention has a protective activity of immature, normal or mature/aged skin and/or mucous membranes (gums, periodontium, genital mucous membranes) and/or skin appendages and/or annexes thereof, against mechanical, microbial, thermal and radical aggressions. The extract according to the invention acts for the defense of the microbiota and therefore allows to fight against the microbiota imbalance. Furthermore, the extract according to the invention allows to stimulate the immune defenses of the skin and the skin antioxidant system.

The invention also relates to the use of an extract according to the invention or of a composition according to the invention for the manufacture of a cosmetic, pharmaceutical or dermatological composition for preventing and/or treating disorders or pathological conditions of immature, normal or mature/aged skin and/or mucous membranes (gums, periodontium, genital mucous membranes) and/or skin appendages (hair and nails), in particular inflammatory reactions, oxidation reactions, disorders related to radical attacks whether or not related to pollution, disorders or pathological conditions related to microbial attacks, barrier or homeostasis disorders, aging, in particular chronological and/or or actinic aging, disorders or pathological conditions related to mechanical and/or thermal aggressions on the skin and/or mucous membranes and/or skin appendages; more advantageously inflammatory, irritative reactions or pathological conditions or disorders of the barrier or homeostasis of immature, normal or mature/aged skin, skin appendages and/or mucous membranes (gums, periodontium, genital mucous membranes).

The invention also relates to the use of an extract according to the invention or a composition according to the invention for the manufacture of a pharmaceutical, cosmetic or dermatological composition for preventing and/or treating imbalances and/or disorders related to the imbalance of the microbiota of immature, normal or mature/aged skin and/or mucous membranes (gums, periodontium, genital mucous membranes) and/or skin appendages and/or annexes thereof.

The invention further relates to a method for preventing and/or treating disorders or pathological conditions of immature, normal or mature/aged skin and/or mucous membranes (gums, periodontium, genital mucous membranes) and/or skin appendages (hair and nails), in particular inflammatory reactions, oxidation reactions, disorders related to radical attacks related or not to pollution, disorders or pathological conditions related to microbial attacks, barrier or homeostasis disorders, aging, in particular chronological and/or actinic aging, disorders or pathological conditions related to mechanical and/or thermal aggressions on the skin and/or mucous membranes and/or skin appendages; more advantageously inflammatory or irritative reactions or pathological conditions or disorders of the barrier or homeostasis of immature, normal or mature/aged skin, skin appendages (hair and nails) and/or mucous membranes (gums, periodontium, genital mucous membranes), comprising the administration, in particular topical administration, of an effective quantity of an extract according to the invention or of a composition according to the invention, to a subject in need thereof.

The invention also relates to a method for preventing and/or treating imbalances and/or disorders related to the imbalance of the microbiota of immature, normal or mature/aged skin and/or mucous membranes (gums, periodontium, genital mucous membranes) and/or skin appendages and/or annexes thereof, comprising the administration, in particular topical administration, of an effective quantity of an extract according to the invention or of a composition according to the invention, to a subject in need.

In particular, the composition or the extract according to the invention is intended for the prevention and/or treatment of inflammatory or irritative reactions or pathological conditions or disorders of the barrier or homeostasis of immature, normal or mature/aged skin, skin appendages (hair and nails) and/or mucous membranes (gums, periodontium, genital mucous membranes).

Advantageously, the inflammatory, irritative reactions, disorders or pathological conditions or disorders of the barrier or homeostasis of the skin are: acne, rosacea or erythrocouperosis, vascular disorders, in particular redness and rosacea, dermatitis diaper, atopic dermatitis, eczema, contact dermatitis, irritative dermatitis, allergic dermatitis, seborrhoeic dermatitis (cradle cap), sensitive skin, reactive skin, dry skin (xerosis), dehydrated skin, the skin with redness, skin erythema, aged or photo-aged skin, photosensitized skin, pigmented skin (melasma, post-inflammatory pigmentation . . . ), skin with stretch marks, sunburn, irritation by chemical, physical (for example tension stress for pregnant women), bacteriological, fungal agents, skin aging, in particular photo-aging and disorders related to radical attacks related to chemical or atmospheric pollution, and/or related to exposure to UV or IR.

Advantageously, the inflammatory, irritative reactions, disorders or pathological conditions or disorders of the barrier or homeostasis of the mucous membranes are gingivitis (sensitive gums of newborns, hygiene problems, due to smoking or the like), and irritation of the external or internal male or female genital spheres.

Advantageously, the inflammatory, irritative reactions, disorders or pathological conditions or disorders of the barrier or homeostasis of the skin appendages are brittle nails, fragile nails, fragile hair, brittle hair, dry hair.

Advantageously, the reactions, disorders or pathological conditions related to the imbalance of the microbiota of the skin are atopic dermatitis, eczema, the development of bad underarm odors, the weakening of the skin barrier, acne, psoriasis, and hidradenitis suppurativa.

Advantageously, the reactions, disorders or pathological conditions related to the imbalance of the microbiota of the skin appendages are folliculitis, cradle cap, dandruff, itching of the scalp.

Advantageously, the reactions, disorders or pathological conditions related to the imbalance of the microbiota of the mucous membranes are itching, irritation, candidiasis, and bacterial vaginosis.

The invention also relates to the cosmetic use of an extract according to the invention or of a composition according to the invention, advantageously on healthy subjects, in the treatment and/or prevention of dehydrated skin; the skin with redness; aged or photo-aged skin; photosensitized skin; skin aging, in particular photo-aging; and disorders related to radical attacks related to chemical or atmospheric pollution, and/or related to exposure to UV or IR.

The invention also relates to the cosmetic use of an extract according to the invention or of a composition according to the invention for caring for skin appendages, advantageously on healthy subjects, in particular for the treatment and/or prevention of fragile nails; brittle nails; fragile hair; brittle hair; and dry hair.

The invention also relates to a cosmetic care method for the skin and/or skin appendages and/or mucous membranes, in view of improving the condition and/or the appearance thereof, advantageously on healthy subjects, which method consists in administering a composition or an extract according to the present invention.

In particular, the invention relates to a process for the cosmetic care of the skin and/or skin appendages, in view of preventing the alterations of the barrier and its dehydration, advantageously on healthy subjects, consisting in applying to the skin and/or hair and nails a composition or an extract according to the present invention.

In particular, the invention relates to a cosmetic care method for the skin and/or skin appendages, advantageously on healthy subjects, for preventing and/or treating alterations to the skin barrier; dehydrated skin; the skin with redness; aged or photo-aged skin; photosensitized skin; skin aging, in particular photo-aging; disorders related to mechanical or thermal aggression of the skin and disorders related to radical attacks related to chemical or atmospheric pollution, and/or related to exposure to UV or IR rays, which method consists in administering a composition or one of the extracts according to the present invention.

The following examples illustrate the invention in a non-limiting manner.

### DESCRIPTION OF THE FIGURES

FIG. 1 represents the growth curves of C. acnes and M. furfur (see Example 2-V-b.).

FIG. 2 represents the effect of the BCP active ingredient on the bacterial growth of different strains in co-culture as a function of time (see Example 2-V-b.).

FIG. 3 represents the analysis of the morphology of the RHEs after Hematoxylin/Eosin staining (see Example 2-VI-b.).

FIG. 4a and FIG. 4b represent the growth curves of the lactobacillus strains (see Example 2-VII-b.).

FIG. 5 represents the analysis of the formation of biofilm by different strains of the microbiota of the skin in the presence of the BCP active ingredient (see Example 2-IX).

### EXAMPLES

### Example 1: Preparation of a Solution of Polysaccharides from Calyxes of Bombax costatum

The Bombax costatum Calyxes are ground, then suspended with stirring in water at a proportion of 2% w/w calyxes/water;
Extraction for one hour with stirring at 90° C.;
Centrifugation of the solution obtained in order to separate the solid residues of the plant;
Bleaching by addition of activated carbon and filtration;
Concentrate of polysaccharides by Ultrafiltration 15 kDa;
Addition of glycerin and evaporation of the water under vacuum in order to obtain a final glycerin concentration of 80% w/w.

Analytical profile of the polysaccharide solution obtained
Orange viscous solution
Dry matter (m/m): 1.24%
pH: 6.3
Ash 8.9%
Total sugars (determination by the anthrone method): 35%
Viscosity of the solution (TA, mobile1, 2 rpm): 2592 Cps

Analytical profile of the polysaccharides obtained (dry matter):
Average molecular mass (determination by Gas Phase Chromatography): 3711 kDa
Composition (determination by Gas Phase Chromatography): 15% Galactose; 17% rhamnose; 17% galacturonic acid; 5% glucuronic acid

### Example 2: Biological Activities

The potential biological activities of the extract were investigated by a gene expression modulation test on dermal fibroblasts and melanized reconstructed epidermis. Thus, the expression of 96 genes of major interest in skin and cosmetic physiology was studied by PCR-array on fibroblasts and melanized reconstructed epidermis.

a. Materials and Methods:

The Bombax costatum polysaccharide extract according to Example 1 (called BCP extract) at 0.05% dry matter was added to the culture medium of normal human dermal fibroblasts (NHDFs) or of reconstituted melanized human epidermis.

After 6 hours or 24 hours of incubation, the expression of the selected markers was evaluated by quantitative RT-PCR (TaqMan microfluidic card). The variation in expression of the markers studied compared to the control was expressed in relative quantity (RQ, RQ>1: increase, RQ<1: decrease).

b. Results:

The most significant results showing the effect of BCP extract on gene expression in reconstructed epidermis are shown in Table 1 below.

TABLE 1
Variations in the expression of genes of interest
in melanized reconstructed human epidermis

| BCP 0.05% dm | RQ | p value |
|---|---|---|
| RAB11A Ras-related protein Rab-11A | 1.7372 | 0.0073 |
| SMPD1 Sphingomyelin phosphodiesterase | 1.4885 | 0.0012 |
| (Acid sphingomyelinase) | | |
| KRT19 Keratin, type I cytoskeletal 19 | 1.3016 | 0.0321 |
| (keratin 19) | | |
| SDC1 Syndecan-1 | 1.279 | 0.0401 |
| MITF Microphthalmia-associated | 0.7409 | 0.0298 |
| transcription factor | | |

Relative Quantity (RQ) compared to the control = 1
p value determined following a Student test

These results show that the BCP extract, by varying the gene expression of some markers, has a particular interest in the following activities:

Synthesis and Remodeling of Lipids in the Barrier Function of the Epidermis

The BCP extract increases the gene expression of 2 enzymes involved in the synthesis or remodeling of lipids within the stratum corneum: The RAB11A gene for a GTPase (Ras-related protein Rab-11A) and the SMPD1 gene for sphingomyelin phosphodiesterase. By increasing the expression of these two genes, the BCP active ingredient allows to reinforce the barrier function of the epidermis and its hydration.

Mechanisms Regulating Cell Adhesion, Migration, Proliferation and Differentiation of Keratinocytes:

The BCP active ingredient increases the expression of syndecan-1 (SDC1).

Stem Cell Protection:

Keratin 19 encoded by the KRT19 gene is an epithelial marker considered as a marker of epidermal stem cells.

By stimulating the expression of KRT19, the BCP extract has a protective effect on stem cells.

Inhibition of Melanogenesis:

The BCP extract causes a decrease in the expression of the MITF gene. Thus, by decreasing the expression of the MITF gene, the BCP extract has melanogenesis inhibitory activity.

Table 2 below shows the most significant results of the BCP extract on gene expression in fibroblasts.

TABLE 2
Variations in the expression of genes of interest
in normal human dermal fibroblasts (NHDFs)

| BCP 0.05% dm | RQ | p value |
|---|---|---|
| SOD2 Superoxide dismutase 2, | 9.4811 | 0.0045 |
| mitochondrial | | |
| MT1G Metallothionein-1G | 5.1496 | 0.0037 |
| FBN2 Fibrillin-2 | 2.9182 | 0.026 |
| FOXO1 Forkhead box protein O1 | 2.0353 | 0.0118 |
| CSGALNACT1 Chondroitin sulfate N- | 2.0195 | 0.0055 |
| acetylgalactosaminyltransferase 1 | | |
| MKI 67 Antigen Ki-67 | 1.9088 | 0.0062 |
| DCN Decorin (PGS2) | 1.9083 | 0.0332 |
| CTGF Connective tissue growth factor | 1.8602 | 0.0377 |
| TXNRD1 Thioredoxin reductase 1 | 1.664 | 0.0148 |
| ELN Elastin (tropoelastin) | 1.6088 | 0.0226 |
| LMNB1 Lamin-B1 | 1.5846 | 0.0068 |
| FGF2 Heparin-binding growth factor 2 | 1.5844 | 0.0355 |
| (Fibroblast growth factor 2) | | |
| FBN1 Fibrillin-1 (Marfan syndrome) | 1.4919 | 0.0119 |
| COL3A1 Collagen 3 alpha 1 subunit | 1.4364 | 0.0362 |
| (COL3A1) | | |
| CAT Catalase | 1.2865 | 0.0387 |
| ACTA2 Actin, aortic smooth muscle | 1.2722 | 0.0293 |
| CHST15 Carbohydrate sulfotransferase 15 | 1.2604 | 0.0194 |
| FBLN5 Fibulin-5 | 1.2554 | 0.0391 |
| COL16A1 Collagen 16 alpha 1 subunit | 1.2117 | 0.0422 |
| (COL16A1) | | |
| FTH1 Ferritin heavy chain | 1.205 | 0.0242 |
| PXN Paxillin | 1.1758 | 0.0184 |
| TLN Talin-1 | 1.1482 | 0.0275 |
| SESN2 Sestrin-2 | 0.5513 | 0.0406 |
| MMP1 Matrix metalloproteinase 1 | 0.5478 | 0.0045 |
| (interstitial collagenase) | | |
| PTGS2 Prostaglandin G/H synthase 2 | 0.3023 | 0.0314 |

Relative Quantity (RQ) compared to the control = 1
p value determined following a Student test

These results show an activity of the BCP extract in the following areas:

Protection Against Oxidative Stress:

The BCP extract induces the expression of several enzymes involved in antioxidant defense: superoxide dismutases, in particular superoxide dismutase 1 (Cu/ZnSOD) encoded by the SOD1 gene and superoxide dismutase 2 (MnSOD) encoded by the SOD2 gene.

The BCP active ingredient also induces the expression of one of the 2 isoforms of metallothionein 1 (MT1G).

The BCP active ingredient also induces the TXNRD1 gene encoding for the isoform 1 of thioredoxin reductase.

The joint induction of these genes coding for proteins with antioxidant and/or detoxifying activity confers protection against UVs and heavy metals from, for example, urban pollution.

The Extracellular Matrix and the Elasticity of the Skin for an Anti-Aging Effect:

The main function of fibroblasts present in the dermis is to produce, degrade, and therefore regulate the components of the extracellular matrix (ECM) with which they interact. The ECM is a complex structure formed by a network of collagen fibers, elastin fibers and structural glycoproteins.

Among the ECM proteins regulated by the BCP active ingredient, it is possible to find elastin (ELN) but also fibrillins-1 and -2 encoded respectively by the FBN1 and FBN2 genes. In addition to the induction of ELN and FBN1 genes, the BCP active ingredient also induces FBN2, also constituting the microfibrils.

In parallel with its action on the elastic fibers, the BCP active ingredient also acts on the collagen fibers since it increases the expression of the alpha 1 subunit of collagen 3 (COL3A1).

The joint action of BCP on the expression of the constituents of elastin and collagen fibers as well as on MMP1 and paxillin goes in the direction of an effect on the elasticity of the skin, which is particularly interesting in an anti-aging context.

Moreover, it has been demonstrated that skin aging is associated with a decrease in the proliferation of fibroblasts. There is a decrease in expression of the proliferation factor Ki-67 related to age (Ma, C. and al., 2011. Expression of metallothionein-I and II in skin ageing and its association with skin proliferation. Br. J Dermatol., 164(3), pp. 479-482). The BCP active ingredient increases the expression of MKI67, indicating an increase in cell proliferation, which reinforces its anti-aging effect.

The Nuclear Lamin:

It was shown that the expression of lamin B1 (LMNB1) in the skin decreases with age. Lamin B1 is thus considered to be a marker of skin cell senescence. Indeed, it has been shown that senescence related to age or associated with skin pathological conditions is accompanied by a loss of expression, at the protein and mRNA level, of lamin B1 (Dreesen, O. and al., 2013. The contrasting roles of lamin B1 in cellular aging and human disease. Nucleus, 4(4), pp. 283-290).

Consequently, an increase in the expression of LMNB1 within dermal fibroblasts indicates an effect of the BCP active ingredient reducing senescence which may be associated with age or skin pathological conditions, and on the other hand proves to be beneficial for maintaining core structure and integrity.

Autophagy:

The BCP extract induces a decrease in the expression of SESN2. SESN2 in particular is involved in the UV response of skin cells. This goes in the direction of a greater activity of the mTORC1 complex which goes hand in hand with a decrease in the autophagic activity of the cells and thus a regulatory effect of the mitophagic activity.

Skin Healing:

Within focal adhesion sites, integrins are bonded, via adhesion proteins, to intracellular actin filaments. Among these cytoplasmic adhesion proteins, it is in particular possible to find talin-1 encoded by the TLN1 gene overexpressed by the BCP active ingredient. It constitutes the initial bond between integrins and the actin cytoskeleton. The binding of talin to integrins regulates their affinities for the ECM, whereas the binding of talin to actin constitutes the first link to the contractile machinery of cells. These 2 events are particularly important for cell migration. Indeed, this involves the cyclic attachment and detachment of integrins to the ECM but also the generation of a force required for the translocation of cellular contents (Atherton, P. and al., 2015. Vinculin controls talin engagement with the actomyosin machinery (Nat. Commun, Volume 6, p. 10).

The increase in the expression of the talin-1 gene combined with that of paxillin demonstrates a beneficial action on cell migration, particularly in the skin healing process.

PTGS2 Underexpression in an Anti-Inflammatory Context:

Prostaglandin-endoperoxide synthases, including cyclooxygenase-2 COX2 or PTGS2, catalyze the biosynthesis of prostaglandins (PG) from arachidonic acid in order to induce the inflammatory process via the secretion of cytokines and skin vasodilation.

The decrease in COX2 expression indicates the ability of the BCP active ingredient to reduce a possible inflammatory phenomenon that can be induced by different stimuli such as pathogens, UVs, ionizing radiation, etc.

I. Anti-Inflammatory Activity

The anti-inflammatory activity of the Bombax costatum polysaccharide extract according to Example 1 has been demonstrated with respect to an inflammation induced by PMA stress.

a) Materials and Methods

Normal Human Keratinocytes (NHK) were treated for 24 hours at 37° C. with the Bombax costatum Polysaccharide extract according to Example 1 (called BCP extract) at 0.01% and/or 0.05% of dry matter (DM) or by Dexamethasone or Indomethacin at 10-7M (anti-inflammatory reference molecules). The cells were then treated by adding PMA at 10 μg/ml (Phorbol Myristate Acetate, inflammation-inducing agent) for 16 hours at 37° C. At the end of the treatment, the quantities of IL1α (interleukin 1α), PGE2 (Prostaglandin-E2), IL1β (Interleukin 1β), TNFα (Tumor Necrosis Factor α), IL6 (Interleukin 6) and IL8 (Interleukin 8) secreted were measured by the ELISA technique in the culture supernatants.

The significance of the results was assessed by a one-way analysis of variance followed by a Tuckey test.

b) Results

As shown by the results in Tables 3 to 8, the BCP extract significantly inhibited the production of the inflammatory mediators IL1α, IL1β, IL6, IL8, TNFα and PGE2 in keratinocytes under inflammatory conditions.

These results show the anti-inflammatory activity of the BCP extract.

TABLE 3
Dosage of IL1-alpha produced by NHKs
under the effect of PMA stress

| | IL1α (pg/ml) | | | |
|---|---|---|---|---|
| | | Standard | | |
| | Average | deviation | Evolution | Significance |
| Control | 33.869 | 2.340 | | |
| PMA 10 μg/ml | 372.604 | 91.220 | +1000% | $$ |
| Dexamethasone 0.1 μM | 230.583 | 29.293 | −38% | ns |
| BCP 0.05% dm | 116.557 | 25.046 | −69% | * |

$$ p < 0.01 vs Control;
* p < 0.05
**p < 0.01 vs PMA;
ns Not Significant
One-way ANOVA followed by a Tukey test

TABLE 4
Dosage of IL1-beta produced by NHKs
under the effect of PMA stress

| | IL1β (pg/ml) | | | |
|---|---|---|---|---|
| | | Standard | | |
| | Average | deviation | Evolution | Significance |
| Control | 18.291 | 2.304 | | |
| PMA 10 μg/ml | 50.392 | 6.609 | +175% | $$ |
| Dexamethasone 0.1 μM | 34.410 | 6.003 | −32% | ns |
| BCP 0.05% dm | 24.459 | 4.956 | −51% | * |

$$ p < 0.01 vs Control;
* p < 0.05 vs PMA;
ns Not Significant
One-way ANOVA followed by a Tukey test

TABLE 5
Dosage of IL6 produced by NHKs under the effect of PMA stress

| | IL6 (pg/ml) | | | |
|---|---|---|---|---|
| | | Standard | | |
| | Average | deviation | Evolution | Significance |
| Control | 5.225 | 0.849 | | |
| PMA 10 μg/ml | 6.958 | 0.721 | +33% | $ |
| Dexamethasone 0.1 μM | 2.090 | 0.300 | −70% | *** |
| BCP 0.01% dm | 5.379 | 0.318 | −23% | * |

$ p < 0.05 vs Control;
* p < 0.05
*** p < 0.001 vs PMA
One-way ANOVA followed by a Tukey test

TABLE 6
Dosage of IL8 produced by NHKs under the effect of PMA stress

| | IL8 (pg/ml) | | | |
|---|---|---|---|---|
| | | Standard | | |
| | Average | deviation | Evolution | Significance |
| Control | 161.769 | 10.601 | | |
| PMA 10 μg/ml | 9952.388 | 1393.896 | 6052 | $$$ |
| Dexamethasone 0.1 | 4076.675 | 202.317 | −59 | *** |
| μM | | | | |
| BCP 0.01% dm | 6641.050 | 804.243 | −33 | * |

$$$ p < 0.001 vs Control;
* p < 0.05
*** p < 0.001 vs PMA;
ns Not Significant
One-way ANOVA followed by a Tukey test

TABLE 7
Dosage of TNF-alpha produced by NHKs
under the effect of PMA stress

| | TNFα (pg/ml) | | | |
|---|---|---|---|---|
| | | Standard | | |
| | Average | deviation | Evolution | Significance |
| Control | 1.546 | 0.655 | | |
| PMA 10 μg/ml | 33.787 | 0.785 | +2086% | $$$ |
| Dexamethasone 0.1 μM | 16.216 | 1.633 | −52% | *** |
| BCP 0.01% dm | 12.991 | 0.600 | −62% | *** |
| BCP 0.05% dm | 21.546 | 0.936 | −36% | * |

$$$ p < 0.001 vs Control;
* p < 0.05
*** p < 0.001 vs PMA
One-way ANOVA followed by a Tukey test

TABLE 8
Dosage of PGE2 produced by NHKs
under the effect of PMA stress

| | PGE2 (pg/ml) | | | |
|---|---|---|---|---|
| | | Standard | | |
| | Average | deviation | Evolution | Significance |
| Control | 39.000 | 0.000 | | |
| PMA 10 μg/ml | 625.506 | 109.911 | +1504% | $$$ |
| Dexamethasone 0.1 μM | 39.000 | 0.000 | −94% | *** |
| BCP 0.05% dm | 219.335 | 108.511 | −65% | ** |

$$$ p < 0.001 vs Control;
** p < 0.01
*** p < 0.001 vs PMA;
ns Not Significant
One-way ANOVA followed by a Tukey test

II. Activity on Epidermal Healing.

The potential activity on epidermal healing of the Bombax costatum Polysaccharide extract according to Example 1 (called BCP extract) was evaluated by studying keratinocyte migration, the first step in the process of skin re-epithelialization.

a) Materials and Methods

Scratch Assay: a lesion was performed on a mat of normal human keratinocyte monolayer (NHK) at confluence before treatment with BCP extract at 0.01% and 0.05% dry matter or with EGF (Epidermal Growth Factor) at 100 ng/ml.

A photo was taken at the start of treatment and after 5 hours of incubation in order to measure the rate of progression of the HCNs within the lesion. The percentage of coverage was evaluated under the different conditions by image analysis.

The significance of the results was assessed by Student's t test.

b) Results

As demonstrated by the results in Table 9, the BCP extract significantly stimulated keratinocyte migration, thus confirming its potential for activating skin healing.

TABLE 9
Migration of NHK

| | | Evolution compared |
|---|---|---|
| | % recovery | to the control |
| Control | 33.6 ± 3.1 | 0 |
| EGF 100 ng/ml | 62.2 ± 11.1 | +85% ** |
| BCP 0.01% dm | 55.4 ± 14.1 | +65% * |
| BCP 0.05% dm | 67.0 ± 8.6 | +99% *** |

* p < 0.05
** p < 0.01
*** p < 0.001 vs control; t test

III. Activity on Innate Anti-Microbial Defenses

The effect of the Bombax costatum Polysaccharide extract according to Example 1 (called BCP extract) was evaluated on the expression of anti-microbial peptides (AMPs) and of TLR2 in epidermal keratinocytes.

a) Materials and Methods

Normal human epidermal keratinocytes were treated for 24 or 48 hours with BCP extract or IL1β at 100 ng/ml (positive reference).

At the end of the treatment, the gene expression of hBD2, hBD3 and TLR2 was analyzed by real-time quantitative RT-PCR.

Moreover, the quantities of TLR2 and intracellular hBD2 were measured by ELISA.

b) Results

As demonstrated by the results in Tables 10 and 11, the BCP extract significantly stimulated the gene and protein expression of defensins and TLR2; thus demonstrating an anti-microbial activity and activation of immune defenses.

TABLE 10
Gene expression of PAMs and TLR2 in keratinocytes
(RQ: Relative Quantity)

| | hBD2 | | hBD3 | | TLR2 | |
|---|---|---|---|---|---|---|
| | RQ | Evolution | RQ | Evolution | RQ | Evolution |
| Control | 1.12 | | 1.09 | | 1.08 | |
| IL1β 100 ng/ml | 3480.89 | ×3000 *** | 9.04 | +730% *** | 3.26 | +201% *** |
| BCP 0.01% dm | 12.94 | +1057% *** | 2.69 | +147% ns | 2.14 | +98% * |
| BCP 0.05% dm | 293.91 | ×300 *** | 10.69 | +882% *** | 2.81 | +160% ** |

* p < 0.05;
** p < 0.01;
*** p < 0.001;
ns Not Significant vs Control
One-way ANOVA followed by Dunnett test

TABLE 11
Production of hBD2 and TLR2 in keratinocytes
(Intracellular protein expression)

| | hBD2 | | TLR2 | |
|---|---|---|---|---|
| | pg/ml | Evolution | pg/ml | Evolution |
| Control | 141.5 ± 15.2 | | 735.9 ± 39.2 | |
| IL1β 100 | 2162.7 ± 51.1 | +1428% *** | 913.7 ± 30.5 | +24% ** |
| ng/ml | | | | |
| BCP 0.006% | 286.33 ± 66.1 | +102% * | 789.2 ± 78.2 | +7% ns |
| dm | | | | |
| BCP 0.02% dm | 488.8 ± 57.3 | +245% *** | 884.2 ± 56.0 | +20% * |

* p < 0.05;
** p < 0.01;
*** p < 0.001;
ns Not Significant vs Control
One-way ANOVA followed by Dunnett test

IV. Activity on Bacterial Adhesion

The effect of the BCP extract was evaluated on the adhesion of 4 bacterial strains to the surface of reconstructed human epidermis (RHE) or skin explants.

a) Materials and Methods
Study of the adhesion of S. aureus, S. epidermidis and C. acnes:

Human Reconstructed Epidermis (RHE) were incubated for 15 min in the presence of the Bombax costatum polysaccharide extract according to Example 1 (called BCP extract) at 0.6% and 2%. Then, after two rinsings with PBS, the bacterial strains Staphylococcus epidermidis (ATCC 14990), Staphylococcus aureus (ATCC 6538) or Cutibacterium acnes (ATCC 6919) were deposited on the surface of the RHEs for 4 hours. After elimination of non-adherent bacteria by 4 successive washes, the RHEs were again incubated overnight.

The counting of the bacteria was carried out by counting the colonies after seeding on specific agar of the ground RHE. The result is expressed in CFU/RHE (colony forming unit).
Study of the adhesion of C. xerosis:

Skin explants were incubated for 2 hours in the presence of BCP extract at 0.6% and 2%. A suspension of Corynebacterium xerosis (DSM 20743) was then deposited on the surface of the skin explants for 2 hours. After rinsing, these explants were again incubated for 24 hours.

The bacteria adhering to the surface of the skin explants were recovered by scraping then seeded on agar in order to carry out the count expressed in CFU/cm².

b) Results

At the two concentrations tested, the BCP extract induced a marked inhibition of the adhesion of S. aureus on RHEs. At 2%, the BCP extract also inhibited the adhesion of C. acnes. (See table 12)

The BCP extract significantly inhibited the adhesion of C. xerosis at the surface of explants (see Table 13).

The BCP extract promotes a rebalancing of the skin microbiota by limiting the adhesion of pathogenic bacteria while preserving the adhesion of commensal bacteria.

TABLE 12
Bacterial adhesion on RHE

| | C. acnes | | S. aureus | | S. epidermidis | |
|---|---|---|---|---|---|---|
| | | Evo- | | Evo- | | Evo- |
| | UFC/RHE | lution | UFC/RHE | lution | UFC/RHE | lution |
| Control | 23125 | | 1275000 | | 2764801 | |
| BCP 0.6% | NC | NC | 137875 | −89% | 2650852 | −4% |
| BCP 2% | 8125 | −65% | 244750 | −81% | 2146464 | −22% |

TABLE 13
Adhesion of C. xerosis on skin explants

| | UFC/cm² | Evolution | Significance |
|---|---|---|---|
| Control | 597 ± 190 | | |
| BCP 0.6% | 272 ± 114 | −54% | ** |
| BCP 2% | 48 ± 38 | −92% | *** |

** p < 0.01;
*** p < 0.001 - one-way ANOVA followed by Dunnett test

V. Activity on Bacterial Growth

The antimicrobial or, on the contrary, prebiotic activity of the Bombax costatum polysaccharide extract according to Example 1 (called BCP extract) was evaluated by studying the bacterial growth of different strains cultured separately or in co-culture.

a) Materials and Methods

The following microbial strains, representative of the skin microbiota, were used:
Staphylococcus aureus (ATCC 6538);
Staphylococcus epidermidis (ATCC 12228);
Cutibacterium acnes (ATCC 11827);
Corynebacterium xerosis (ATCC 373);
Malassezia furfur (ATCC 14521);
Staphylococcus hominis (ATCC 27844).
Determination of the minimum inhibitory concentration (MIC) and determination of the “activator” potential of bacterial growth:

The BCP extract was diluted in microplates at 0.25%; 0.5%; 1%; 2%; 4% and 8%, in the minimal culture medium specific to each strain. The controls were prepared similarly to the sample:
A positive growth inhibition control: Phenonip® 5% in the diluted medium,
A control consisting of diluted growth medium,
A control consisting of growth medium.

The strains were added at the different conditions before incubation for 48 hours.

In the presence of a disorder revealing microbial growth after 48 hours, an aliquot was taken for the three highest concentrations still showing growth of microorganisms. These aliquots were deposited in separate plates in decimal dilutions, so as to determine the concentration of the populations by limiting dilutions.

After 48 hours of incubation, the activation of bacterial growth was demonstrated by determining the concentration of each microorganism (carried out simultaneously by visual reading and measurement of absorbance at 620 nm (turbidity)) according to decimal dilutions carried out.

Finally, the results were expressed by comparison with the bacterial growth obtained in the controls without product.
Determination of the effect on the growth of bacteria in co-culture:

The S. aureus, S. epidermidis, S. hominis and C. acnes strains in co-culture were incubated for 48 hours in the presence of the 0.25% BCP extract; 0.5% and 1% or Phenonip 0.5%, positive growth inhibition control.

An aliquot was taken and placed on agars specific to the selected strains. After 48 hours of incubation, counting was performed for each strain.

b) Results
Determination of the minimum inhibitory concentration (MIC) and determination of the “activator” potential of bacterial growth:

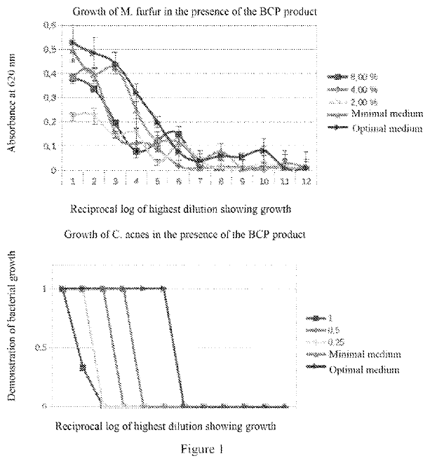

On C. acnes, an inhibition of bacterial growth was observed with a MIC determined at 4%. The growth kinetics showed a bacteriostatic effect of the BCP extract against C. acnes (see FIG. 1).

Similarly, the growth kinetics of M. furfur showed a bacteriostatic effect of the BCP extract against this yeast (see FIG. 1).
Determination of the effect on the growth of bacteria in co-culture:

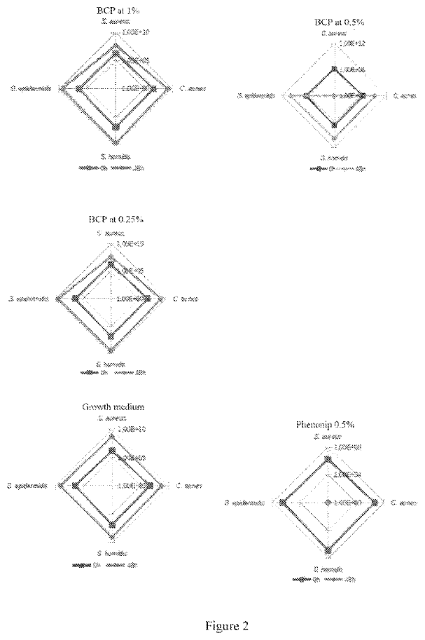

The BCP extract showed a nourishing effect against S. epidermidis, S. hominis and C. acnes strains and tended to inhibit the growth of S. aureus (bactericidal effect) (see FIG. 2).

These results show a protective effect on the balance of the skin microbiota.

VI. Defense Against S aureus Induced Damage

The bacterium Staphylococcus aureus (S. aureus) is frequently detected in patients with atopic dermatitis.

The quantity of S. aureus present in these patients is correlated with the degree of severity of the pathological condition and plays an important role in the pathophysiology.

The objective of this study was to reproduce the stress caused by this bacterium on the epidermis, for this purpose secretum of S. aureus was applied to the surface of RHE in order to study the consequences on proteins of epidermal differentiation.

a) Materials and Methods

The BCP extract according to Example 1 at 1% was applied as a 24-hour pre-treatment to the surface of reconstructed human epidermis (RHE). The RHEs were then treated topically by depositing the secretum (culture medium) of Staphylococcus aureus (ATCC 33592).

After an additional 24 hours of incubation, a morphological analysis of the tissues was carried out after Hematoxylin/Eosin staining, as well as the analysis of the expression of barrier function markers by immunofluorescence. The level of expression of the proteins of interest was evaluated by semi-quantification using Image J software.

b) Results

Morphological Analysis:

As shown in FIG. 3, the secretum of S. aureus induced a moderate alteration in the morphology of the RHE: disorganization of the structure of the epidermis (basal layer), fewer grains of keratohyaline at the granular layer.

The pre-treatment with the BCP extract preserved the morphology of the epidermis which are better organized, thicker and with a more marked production of keratohyaline grains.

Expression of Barrier Function Markers:

As demonstrated by the results in Table 14, S. aureus secretum significantly reduced the level of expression of the barrier function markers studied: Corneodesmosin, Desmoglein-1 and Filaggrin. This model is therefore representative of the negative impact of S. aureus on the barrier function, in particular in the pathophysiology of atopic dermatitis.

The BCP extract allowed to counterbalance this decrease in expression.

TABLE 14
Expression of barrier markers in RHEs

| | Corneodesmosine | Desmoglein1 | Filaggrin |
|---|---|---|---|
| Control | 100 | 100 | 100 |
| Secretum S. aureus | 49.6 $ | 35.4 $$ | 42.5 $$ |
| Secretum + BCP 1% | 84.2 * | 120.1 ** | 79.4 * |

$ p < 0.05;
$$ p < 0.01 vs Control;
* p < 0.05;
** p < 0.01 vs secretum - Unpaired t test

The BCP extract could therefore protect the skin from aggression by S. aureus.

VII. Protective Activity of the Vaginal Microflora

The prebiotic efficacy of the BCP extract according to Example 1 was evaluated on 3 strains of Lactobacilli representative of the vaginal tract; moreover, the BCP extract was studied in a reconstructed model of vaginal epithelium colonized by strains of lactobacilli representative of the resident microflora.

### a) Materials and Methods

Study of the prebiotic effect on L. gasseri, L. acidophilus, L. rhamnosus:

The L. gasseri (ATCC 33323), L. acidophilus (LA-14) and L. rhamnosus (ATCC 53103) strains were inoculated in a depleted medium in the presence of the Bombas costatum polysaccharide extract according to Example 1 (called BCP extract) at 0.25%; 0.5%; 1% and 2% or with addition of 2% glucose (positive control).

After 4 h and 24 h of incubation, the bacterial growth was evaluated by spectrophotometric measurement of the OD at 600 nm (which gives an indication of the rate of bacterial replication), as well as the bacterial viability expressed in CFU/ml (to quantify the number of viable residual bacteria).

The influence on bacterial metabolism was evaluated by measuring the production of lactic acid in the culture supernatants after 24 hours of incubation.
Study of the protective effect on reconstituted vaginal epithelium:

The 1% BCP extract was applied to the surface of a reconstructed human vaginal epithelium (HVE), at the same time, the epithelia were colonized by a mixture of L. crispatus (DSM 20356) and L. gasseri (DSM 20243), mimicking the physiological resident microflora.

After 6 hours of incubation, the gene expression of the anti-microbial peptide hBD2 was evaluated by real-time RT-PCR.

b) Results
Study of the prebiotic effect on L. gasseri, L. acidophilus, and L. rhamnosus

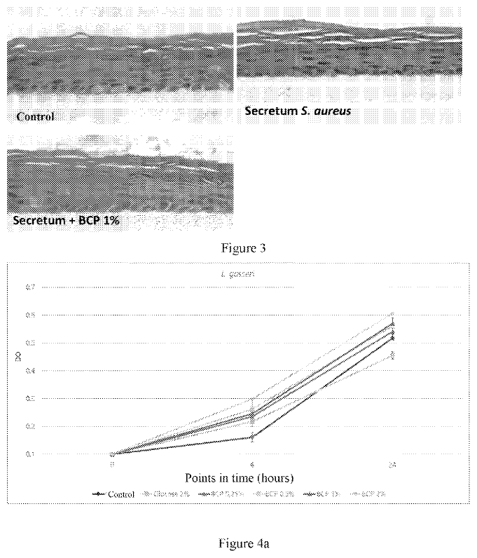

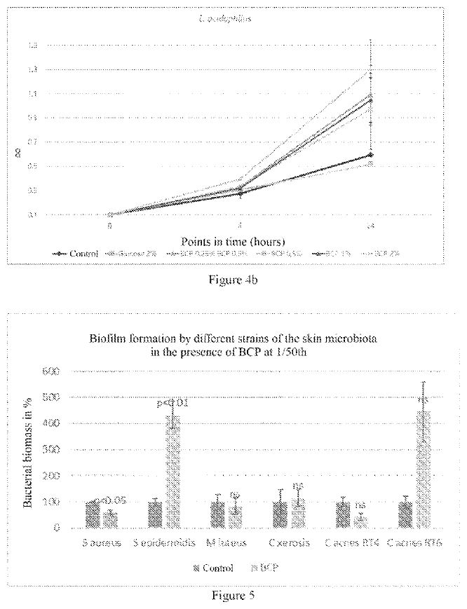

The bacterial growth rate evaluated by measuring the DO showed a positive effect of the BCP extract on the growth of L. gasseri, in a dose-dependent manner from 4 hours (early prebiotic effect); as well as on L. acidophilus, with more particularly a prebiotic effect after 24 hours of incubation (stationary phase) (see FIGS. 4a and 4b).

Overall, the BCP extract had a similar or even greater effect than the positive control (glucose) on the viability of lactobacilli, confirming its prebiotic effect.

Evaluation of bacterial viability (see Table 15) showed that the 2% BCP extract showed the greatest prebiotic effect at 4 hours on the 3 strains of lactobacilli, in particular L. gasseri and, to a lesser extent, L. rhamnosus. This tendecy continues up to 24 hours for L. rhamnosus, while a significant drop in viability was observed at 24 hours for L. gasseri, which is explained by starvation related to culture in an impoverished medium.

At 0.25% the BCP extract induced a prebiotic effect on L. acidophilus.

TABLE 15
Bacterial viability of lactobacillus strains

| | L. gasseri | | L. acidophilus | | L. rhamnosus | |
|---|---|---|---|---|---|---|
| | (Log10 CFU/ml) | | (Log10 CFU/ml) | | (Log10 CFU/ml) | |
| | 4 h | 24 h | 4 h | 24 h | 4 h | 24 h |
| Control | 7.98 ± | 3.10 ± | 7.77 ± | 5.84 ± | 7.82 ± | 8.23 ± |
| | 0.01 | 0.28 | 0.13 | 0.09 | 0.21 | 0.00 |
| Glucose | 7.81 ± | 3.42 ± | 7.73 ± | 7.22 ± | 7.83 ± | 9.09 ± |
| 2% | 0.27 | 0.14 | 0.23 | 0.15 | 0.06 | 0.07 |
| BCP | 7.89 ± | 2.49 ± | 7.72 ± | 8.99 ± | 7.77 ± | 8.89 ± |
| 0.25% | 0.14 | 0.12* | 0.13 | 0.55* | 0.10 | 0.27 |
| BCP | 8.01 ± | 0.00 ± | 7.74 ± | 6.10 ± | 7.83 ± | 8.51 ± |
| 0.5% | 0.19 | 0.00** | 0.08 | 0.57 | 0.06 | 0.56 |
| BCP | 8.02 ± | 3.27 ± | 8.10 ± | 6.29 ± | 7.74 ± | 7.82 ± |
| 1% | 0.10 | 0.38 | 0.58 | 0.02 | 0.06 | 0.01* |
| BCP | 10.40 ± | 3.73 ± | 8.83 ± | 6.63 ± | 8.75 ± | 10.35 ± |
| 2% | 0.24** | 0.07 | 0.18 | 0.25 | 0.11** | 0.04* |

*p < 0.05;
**p < 0.01 - ANOVA test

Lactic acid, a product of the primary metabolism of lactobacilli, was quantified in L. Acidophilus culture media (see Table 16).

The BCP extract induced an increase in lactic acid production by L. acidophilus.

TABLE 16
Quantification of lactic acid produced by L. acidophilus

L. acidophilus

| | Lactic Acid | |
|---|---|---|
| | (nmol/μl) | Evolution |
| Control | 25421.81 ± 4389.106 | |
| Glucose 2% | 24200.96 ± 1062.115 | −5% |
| BCP 0.25% | 57556.58 ± 29969.59 | +126% |
| BCP 0.5% | 46584.36 ± 2749.858 | +83% |
| BCP 1% | 48314.47 ± 12267.67 | +90% |
| BCP 2% | 90768.17 ± 945.7198 | +257% |

*p < 0.05;
**p < 0.01 - ANOVA test

Overall, these results show a prebiotic effect of the BCP extract against L. acidophilus. This effect is greater at a concentration of 2%.

TABLE 17
Summary of the prebiotic effect on lactobacilli

| | Growth rate | | Viability | | Metabolism |
|---|---|---|---|---|---|
| | 4 h | 24 h | 4 h | 24 h | (Lactic acid) |
| | (exponen- | (station- | (exponen- | (station- | 24 h |
| | tial | ary | tial | ary | (station- |
| | phase) | phase) | phase) | phase) | ary phase) |
| L. gasseri | ++ | + | ++ | + | + |
| L. | + | ++ | + | ++ | ++ |
| acidophilus | | | | | |

++ greater effect than positive control; high prebiotic effect
+ effect equivalent to the positive control; prebiotic effect
− no change compared to the control; lack of prebiotic effect

Study of the protective effect on reconstituted vaginal epithelium (HVE):

In the colonized vaginal epithelium (HVE) model, the BCP extract significantly increased hBD2 gene expression (see Table 18).

This result shows a booster effect of local epithelial defenses to protect the mucous membrane from the risk of infection.

TABLE 18
Gene expression of hBD2 in HVE colonized by Lactobacilli

| | hBD2 (RQ) |
|---|---|
| Control | 1.00 |
| HVE colonized | 0.88 |
| HVE colonized + BCP 1% | 3.24 |

RQ: Relative Quantity

VIII. In Vitro Sniff Test (Deodorant Activity)

An in vitro “sniff test” was carried out in order to evaluate the ability of the BCP extract according to Example 1 to inhibit or modify the production of odors by the main bacterial species responsible for underarm odors: C. striatum, S. epidermidis.

a) Materials and Methods

Staphylococcus epidermidis (ATCC 12228) and Corynebacterium striatum (ATCC 6940) strains were used.

Each of these strains has a typical olfactory signature, the result of the metabolism of sweat components:
S. epidermidis: Valeric and propionic acid;
C. striatum: Sulphanyl alcohols.

Each bacterial strain was inoculated into a reconstituted sweat solution in the presence of the BCP extract at 0.1%; 0.6% or 2% or 0.1% chlorhexidine digluconate (positive control).

After 6 h and 24 h of incubation:

The bacterial viability was evaluated by bacterial count expressed in CFU/ml.

A sniff test was carried out to determine the olfactory signature qualitatively and semi-quantitatively (intensity of the smell) immediately after opening the vial, the smell was evaluated and scored by a trained operator:
1. Absent (comparable to 0.1% chlorhexidine positive control)
2. Moderate
3. Intense (bacteria-specific olfactory signature)

b) Results

The BCP extract inhibited the odor produced by S. epidermidis and C. striatum compared to the negative control, without altering the viability of these bacteria (see Table 19). These results demonstrate deodorant activity.

TABLE 19
Viability and odor of bacteria in reconstituted sweat

| | 6 h | | 24 h | |
|---|---|---|---|---|
| | Viability | Odor | Viability | Odor |
| | (Log10 CFU/ml) | (Sniff) | (Log10 CFU/ml) | (Sniff) |
| | S. epidermidis | | | |
| Control | 8.91 ± 0.21 | Intense | 8.69 ± 0.09 | Intense |
| (reconstituted sweat) | | | | |
| Chlorhexidine | 0.00 ± 0.00 ** | Absent | 0.00 ± 0.00 ** | Absent |
| BCP 0.1% | 8.80 ± 0.08 | Moderate | 8.64 ± 0.30 | Moderate- |
| | | | | Intense |
| BCP 0.6% | 8.87 ± 0.07 | Moderate- | 8.87 ± 0.15 | Moderate- |
| | | Absent | | Absent |
| BCP 2% | 8.73 ± 0.34 | Moderate | 8.78 ± 0.18 | Moderate- |
| | | | | Absent |
| | C. striatum | | | |
| Control | 8.51 ± 0.08 | Moderate | 8.64 ± 0.19 | Moderate |
| (reconstituted sweat) | | | | |
| Chlorhexidine | 0.00 ± 0.00 ** | Absent | 0.00 ± 0.00 ** | Intense |
| BCP 0.1% | 8.59 ± 0.31 | Moderate | 8.38 ± 0.11 | Moderate |
| BCP 0.6% | 8.66 ± 0.32 | Absent | 8.57 ± 0.11 | Moderate- |
| | | | | Absent |
| BCP 2% | 8.28 ± 0.06 | Absent | 8.31 ± 0.28 | Absent |

** p < 0.01 - ANOVA test vs Control

IX. Study of Bacterial Biofilm

A biofilm is defined as the assembly of microbial cells associated with a living organism or a tissue and “coated” in a polysaccharide matrix.

The biofilm is one of the most important virulence factors in infectious diseases, it confers resistance to antibiotics, protection against host defenses, it increases the virulence of pathogenic bacteria by promoting their communication system (Quorum Sensing).

In the skin, biofilms have been described in various pathological conditions or disorders: acne, rosacea, atopic dermatitis. Biofilms are also likely to disrupt healing mechanisms. Bacteria that associate in biofilm have an innate resistance to antibiotics, disinfectants and host defense systems; biofilms thus promote the persistence of pathogenic bacteria and their recalcitrance to treatment.

For example, the involvement of the Staphylococcus aureus biofilm has been particularly described in the context of the pathophysiology of atopic dermatitis. Indeed, probably due to the alteration of the surface of the skin which allows it to adhere, s. aureus is more easily found in the form of a biofilm in atopic skin; this state of biofilm promotes its resistance to treatment and increases its virulence, which results in the induction of chronic inflammation and pruritus, phenomena that contribute to the vicious circle of the pathogenesis of atopic dermatitis.

The effect of the BCP extract according to the present invention was evaluated on the biofilm-forming capacity of bacteria of the skin microflora.

a) Materials and Methods
Microbial strains tested:
Staphylococcus epidermidis MFP04: healthy human skin strain (LMSM set of strains) characterized by metabolic analysis, 16S RNA and genomic sequencing. It is a strain rather described as commensal.
Staphylococcus aureus MFP03: healthy human skin strain (LMSM set of strains) characterized by metabolic analysis, 16S RNA and genomic sequencing. It is a commensal strain, an “opportunistic” pathogen, which can be responsible for certain skin infections and widely described as being involved in the pathophysiology of atopic dermatitis.
Corynebacterium xerosis CIP 100653T/ATCC373: strain from international databases. It is a commensal strain, involved in the production of underarm odors.
Cutibacterium (propionibacterium) acnes HL045PA1/HM-516 ribotype 4 (RT4): “acneic” strain characterized by Fitz-Gibbon and al. and obtained from an international database. It is a pathogenic strain, involved in the pathogenesis of acne.
Cutibacterium (propionibacterium) acnes HL110PA3/HM-554 ribotype 6 (RT6): “non-acneic” strain characterized by Fitz-Gibbon and al. and obtained from an international database. It is a commensal strain.
Micrococcus luteus 0116: healthy human skin strain (LMSM set of strains) characterized by metabolic analysis, 16S RNA and genomic sequencing. It is a commensal strain.
Pre-study of the effect of the BCP active ingredient on bacterial growth kinetics:

A control study was carried out to verify the absence of effect of the BCP active ingredient on the growth of the 6 strains studied.

The active ingredient diluted to 1/50^{th} was added to the medium from the start of the culture. Bacterial growth was monitored in microplates over 24 h, 48 h or 72 h depending on the strains. Absorbance was measured continuously using a microplate reader/incubator.
Study of the effect of the BCP active ingredient on the formation of biofilm by the crystal violet technique:

The production of biofilm of bacterial species was studied in multi-well plates (96) using the crystal violet staining technique after culture in the presence of the active ingredient for 24, 48 or 72 hours depending on the strains.

The results are expressed as a percentage based on the biofilm formation value in the control medium (without active ingredient). Statistical differences were established using the Mann-Whitney test.

b) Results

The BCP extract of kapok polysaccharides according to the invention did not significantly modify the kinetics of bacterial growth under the conditions of the test.

The study of biofilm production by the different bacterial strains revealed the following effects of the kapok tree polysaccharide extract (see FIG. 5):
significant inhibition of S. aureus biofilm formation;
an induction of S. epidermis biofilm formation;
an absence of significant modulation of the formation of biofilms of M. luteus, C. xerosis or C. acnes RT6 (non-acne strain);
inhibition of the formation of the biofilm of the acne strain C. acnes RT4 (acne strain).

Advantageously, the BCP extract of kapok polysaccharides inhibits the biofilm formation capacity of pathogenic bacterial strains (S. aureus and C. acnes RT6 in particular), while preserving or even promoting the capacity of commensal bacteria to form a biofilm. These results are in favor of the effect of the active ingredient on the preservation of the balance of the microbiota, with in particular a benefit in pathological conditions such as atopic dermatitis or acne.

1.-10. (canceled)

11. A polysaccharide-rich extract from flowers of Bombax costatum, comprising at least 15% of polysaccharides relative to the total weight of the dry extract, the polysaccharides having an apparent molecular mass comprised between 1500 and 6000 kDaltons.

12. The extract according to claim 11, comprising at least 30% by weight of polysaccharides, relative to the total weight of the dry extract.

13. The extract according to claim 11, wherein the polysaccharides comprise monosaccharides and derivatives selected from the group consisting of galactose, rhamnose, galacturonic acid, glucuronic acid, and mixtures thereof.

14. The extract according to claim 11, wherein the polysaccharides comprise 10 to 16% galactose; 10 to 17% rhamnose; 10 to 17% galacturonic acid; and 3 to 7% glucuronic acid.

15. The extract according to claim 11, wherein the extract is obtained by solid/liquid extraction of the flowers of Bombax costatum, in water.

16. A process for preparing a polysaccharide-rich extract from flowers of Bombax costatum comprising at least 15% of polysaccharides relative to the total weight of the dry extract, the polysaccharides having an apparent molecular mass comprised between 1500 and 6000 kDaltons, wherein the process comprises at least one solid/liquid extraction step in water.

17. The process according to claim 16, further comprising the following successive steps:
a) crushing the flowers of Bombax costatum,
b) extracting the flowers crushed in step a) in water;
c) separating the solid phase and the liquid phase obtained in step b) by decantation, and/or centrifugation and/or precipitation and/or successive filtrations.

18. A composition comprising, as active ingredient, a polysaccharide-rich extract from flowers of Bombax costatum, comprising at least 15% of polysaccharides relative to the total weight of the dry extract, the polysaccharides having an apparent molecular mass comprised between 1500 and 6000 kDaltons.

19. A method for preventing and/or treating:
imbalances and/or disorders related to the imbalance of the microbiota of the skin and/or mucous membranes and/or skin appendages, and/or
disorders or pathological conditions of the skin and/or mucous membranes and/or skin appendages,
comprising administering to a subject in need thereof an effective amount of a polysaccharide-rich extract from the flowers of Bombax costatum, comprising at least 15% of polysaccharides relative to the total weight of the dry extract, the polysaccharides having an apparent molecular mass comprised between 1500 and 6000 kDaltons.

20. A cosmetic care method for improving the condition and/or the appearance of the skin and/or skin appendages and/or mucous membranes on healthy subjects, comprising administering a polysaccharide-rich extract from the flowers of Bombax costatum, comprising at least 15% of polysaccharides relative to the total weight of the dry extract, the polysaccharides having an apparent molecular mass comprised between 1500 and 6000 kDaltons.

21. The extract according to claim 11, wherein the polysaccharides comprise a mixture of galactose, rhamnose, galacturonic acid, and glucuronic acid.

22. The process according to claim 16, further comprising the following successive steps:
d) bleaching the liquid phase obtained in step c) using a suitable adjuvant;
e) drying the liquid phase obtained in step c) or d); and
f) physically and microbiologically stabilizing the extract obtained in step c), d) or e).

23. The method according to claim 19, wherein:
the imbalances and/or disorders related to the imbalance of the microbiota of the skin and/or mucous membranes and/or skin appendages are selected from atopic dermatitis, eczema, the development of bad underarm odors, the weakening of the skin barrier, acne, psoriasis, hidradenitis suppurativa, folliculitis, cradle cap, dandruff, itching, irritations, candidiasis and bacterial vaginosis, and
the disorders or pathological conditions of the skin and/or mucous membranes and/or skin appendages are selected from inflammatory reactions, oxidation reactions, disorders related to radical attacks related or not to pollution and/or related to exposure to UV or IR, photosensitized skin, disorders or pathological conditions related to microbial attacks, barrier or homeostasis disorders, aging, disorders or pathological conditions related to mechanical and/or thermal aggressions on the skin and/or mucous membranes and/or skin appendages.

24. The cosmetic care method according to claim 20, for preventing and/or treating alterations to the skin barrier; dehydrated skin; the skin with redness; aged or photo-aged skin; skin aging, in particular photo-aging; disorders related to mechanical or thermal aggressions on the skin and disorders related to radical attacks related to chemical or atmospheric pollution.
